(19) United States
(12) Patent Application Publication
(10) Pub. No.: US 2005/0106226 A1
(43) Pub. Date: May 19, 2005

(54) Pretreatment method and system for enhancing transdermal drug delivery

(76) Inventors: Michel J.N. Cormier, Mountain View, CA (US); WeiQi Lin, Palo Alto, CA (US); Juanita Johnson, Belmont, CA (US); Kofi Nyam, Palo Alto, CA (US)

Correspondence Address: Francis Law Group, 1942 Embarcadero, Oakland, CA, 94606, US

(21) Appl. No.: 10/970,901

(22) Filed: Oct. 21, 2004

Related U.S. Application Data
(60) Provisional application No. 60/514,387, filed on Oct. 24, 2003.

(51) Int. Cl. A61K 9/70

(52) U.S. Cl. 424/449

(57) ABSTRACT

A drug delivery system for delivering a biologically active agent through the skin of a patient comprises (i) a pretreatment patch adapted to be placed on the patient's skin, the pretreatment patch having a backing membrane and a microprojection array, the microprojection array being adhered to the backing membrane, the microprojection array including a plurality of microprojections adapted to pierce the stratum corneum of the patient, the pretreatment patch including a skin template that remains on the patient's skin after the pretreatment patch is applied to and removed from the patient's skin, and (ii) a gel patch having a top and bottom surface, the gel patch including a reservoir containing a hydrogel formulation, the gel patch having a skin contact area in the range of approximately 0.5-30 cm².

### CROSS-REFERENCE TO RELATED APPLICATIONS

This application claims the benefit of U.S. Provisional Application No. 60/514,387, filed Oct. 24, 2003.

### FIELD OF THE PRESENT INVENTION

The present invention relates generally to transdermal drug delivery systems and methods. More particularly, the invention relates to a pretreatment method and system for percutaneous drug delivery that provides extended drug delivery.

### BACKGROUND OF THE INVENTION

Drugs are most conventionally administered either orally or by injection. Unfortunately, many drugs are completely ineffective or have radically reduced efficacy when orally administered since they either are not absorbed or are adversely affected before entering the bloodstream and thus do not possess the desired activity. On the other hand, the direct injection of the drug into the bloodstream, while assuring no modification of the drug during administration, is a difficult, inconvenient, painful and uncomfortable procedure which sometimes results in poor patient compliance.

Hence, in principle, transdermal delivery provides for a method of administering drugs that would otherwise need to be delivered via hypodermic injection or intravenous infusion. Transdermal drug delivery offers improvements in both of these areas. Transdermal delivery, when compared to oral delivery avoids the harsh environment of the digestive tract, bypasses gastrointestinal drug metabolism, reduces first-pass effects, and avoids the possible deactivation by digestive and liver enzymes. Conversely, the digestive tract is not subjected to the drug during transdermal administration. Indeed, many drugs, such as aspirin, have an adverse effect on the digestive tract. However, in many instances, the rate of delivery or flux of many agents via the passive transdermal route is too limited to be therapeutically effective.

The word “transdermal” is used herein as a generic term referring to passage of an agent across the skin layers. The word “transdermal” refers to delivery of an agent (e.g., a therapeutic agent such as a drug or an immunologically active agent such as a vaccine) through the skin to the local tissue or systemic circulatory system without substantial cutting or penetration of the skin, such as cutting with a surgical knife or piercing the skin with a hypodermic needle. Transdermal agent delivery includes delivery via passive diffusion as well as delivery based upon external energy sources including electricity (e.g., iontophoresis) and ultrasound (e.g., phonophoresis). While drugs do diffuse across both the stratum corneum and the epidermis, the rate of diffusion through the stratum corneum is often the limiting step. Further, many compounds, in order to achieve an effective dose, require higher delivery rates than can be achieved by simple passive transdermal diffusion.

Theoretically, the transdermal route of agent administration could be advantageous for the delivery of many therapeutic proteins, because proteins are susceptible to gastrointestinal degradation and exhibit poor gastrointestinal uptake and transdermal devices are more acceptable to patients than injections. However, the transdermal flux of medically useful peptides and proteins is often insufficient to be therapeutically effective due to the relatively large size/molecular weight of these molecules. Often the delivery rate or flux is insufficient to produce the desired effect or the agent is degraded prior to reaching the target site, for example while in the patient's bloodstream.

Transdermal drug delivery systems generally rely on passive diffusion to administer the drug while active transdermal drug delivery systems rely on an external energy source (e.g., electricity) to deliver the drug. Passive transdermal drug delivery systems are more common. Passive transdermal systems typically include a drug reservoir containing a high concentration of drug. The reservoir is adapted to contact the skin which enables the drug to diffuse through the skin and into the body tissues or bloodstream of a patient.

The transdermal drug flux is dependent upon the condition of the skin, the size and physical/chemical properties of the drug molecule, and the concentration gradient across the skin. This low permeability is attributed primarily to the stratum corneum, the outermost skin layer which consists of flat, dead cells filled with keratin fibers (keratinocytes) surrounded by lipid bilayers. This highly-ordered structure of the lipid bilayers confers a relatively impermeable character to the stratum corneum.

Various pretreatment methods and apparatus have thus been employed to enhance the transdermal drug flux. Illustrative are the methods and apparatus disclosed in U.S. Pat. No. 3,918,449, U.S. Pat. No. 5,611,806 and U.S. Pat. No. 5,964,729.

There are, however, numerous drawbacks and disadvantages associated with the disclosed prior art pretreatment methods and apparatus. Among the drawbacks are that most of the devices employ one or more “rolling structures” that are adapted to pierce the skin via manual force. As a result, there are significant variations in the effected (or pretreated) area from patient to patient. Variations in the force applied and, hence, penetration of the piercing elements are also likely by virtue of the differences in strength and/or applied angle of the device from patient to patient.

A further drawback is that the efficacy of the noted methods in enhancing transdermal protein flux has been, and continues to be, limited, at least for the larger proteins, by virtue of their size.

Other systems and apparatus that employ tiny skin piercing elements to enhance transdermal drug delivery are disclosed in European Patent EP 0 407063A1, U.S. Pat. No. 5,879,326, U.S. Pat. No. 3,814,097, U.S. Pat. No. 5,279,54, U.S. Pat. No. 5,250,023, U.S. Pat. No. 3,964,482, Reissue No. 25,637, and PCT Publication Nos. WO 96/37155, WO 96/37256, WO 96/17648, WO 97/03718, WO 98/11937, WO 98/00193, WO 97/48440, WO 97/48441, WO 97/48442, WO 98/00193, WO 99/64580, WO 98/28037, WO 98/29298, and WO 98/29365; all incorporated by reference in their entirety.

The disclosed systems and apparatus employ piercing elements of various shapes and sizes to pierce the outermost layer (i.e., the stratum corneum) of the skin. The piercing elements disclosed in these references generally extend perpendicularly from a thin, flat member, such as a pad or sheet. The piercing elements in some of these devices are extremely small, some having a microprojection length of only about 25-400 microns and a microprojection thickness of only about 5-50 microns. These tiny piercing/cutting elements make correspondingly small microslits/microcuts in the stratum corneum for enhancing transdermal agent delivery therethrough.

The disclosed systems further include an integral reservoir for holding the drug and also a delivery system to transfer the drug from the reservoir through the stratum corneum, such as by hollow tines of the device itself. One example of such a device is disclosed in WO 93/17754, which has a liquid drug reservoir. The reservoir must, however, be pressurized to force the liquid drug through the tiny tubular elements and into the skin. Disadvantages of such devices include the added complication and expense for adding a pressurizable liquid reservoir and complications due to the presence of a pressure-driven delivery system.

It is therefore an object of the present invention to provide a pretreatment method and system for transdermal drug delivery that substantially reduces or eliminates the aforementioned drawbacks and disadvantages associated with prior art drug delivery systems.

It is another object of the present invention to provide a pretreatment method and system for transdermal drug delivery that enhances and extends drug delivery.

It is another object of the present invention to provide a pretreatment apparatus (or patch) that provides a skin template after application that enhances transdermal drug delivery.

It is yet another object of the present invention to provide a transdermal drug delivery system having a hydrogel formulation that facilitates the delivery of drugs at an effective rate.

### SUMMARY OF THE INVENTION

In accordance with the above objects and those that will be mentioned and will become apparent below, the drug delivery system for delivering a biologically active agent through the skin of a patient comprises (i) a pretreatment patch adapted to be placed on the patient's skin, the pretreatment patch having a backing membrane and a microprojection array, the microprojection array being adhered to the backing membrane, the microprojection array including a plurality of microprojections adapted to pierce the stratum corneum of the patient, the pretreatment patch including a skin template that remains on the patient's skin after the pretreatment patch is applied to and removed from the patient's skin, and (ii) a gel patch having a top and bottom surface, the gel patch including a reservoir containing a hydrogel formulation, the gel patch having a skin contact area in the range of approximately 0.5-30 cm².

Preferably, the gel patch includes a formulation membrane that is disposed proximate the gel patch reservoir that is adapted to inhibit migration of enzymes and/or bacteria into the hydrogel formulation.

In a further embodiment of the invention, the pretreatment patch includes a polymeric membrane ring that is disposed between the release liner ring and the skin adhesive ring, wherein the skin template comprises the release liner ring, polymeric membrane ring and skin adhesive ring.

In another embodiment, the pretreatment patch includes a polymeric support membrane disposed between the backing membrane and the microprojection array.

Preferably, the microprojection array has a microprojection density in the range of 10-2000 microprojections/cm² and provides a pretreated skin area in the range of approximately 0.5-30 cm² after the pretreatment patch is applied to the skin of the patient.

In one embodiment of the invention, the pretreated skin area is substantially equal to the gel patch skin contact area. In a further embodiment, the pretreated skin area is greater than the gel patch skin contact area. In another embodiment, the pretreated skin area is smaller than the gel patch skin contact area.

Preferably, the hydrogel formulation comprises a water-based hydrogel. In one embodiment of the invention, the hydrogel formulation comprises a polymeric material and, optionally, a surfactant. In one aspect of the invention, the polymeric material comprises a cellulose derivative. In a further aspect of the invention, the polymeric material is selected from the group consisting of hydroxyethylcellulose (HEC), hydroxypropylmethylcellulose (HPMC), hydroxypropycellulose (HPC), methylcellulose (MC), hydroxyethylmethylcellulose (HEMC), ethylhydroxyethylcellulose (EHEC), carboxymethyl cellulose (CMC), poly(vinyl alcohol), poly(ethylene oxide), poly(2-hydroxyethylmethacrylate), poly(n-vinyl pyrolidone), pluronics, and mixtures thereof. In a further aspect of the invention, the surfactant is selected from the group consisting of Tween 20 and Tween 80.

In a preferred embodiment of the invention, the hydrogel formulation includes at least one biologically active agent, the biologically active agent being selected from the group consisting of small molecular weight compounds, polypeptides, proteins, oligonucleotides, nucleic acids and polysaccharides.

In an alternative embodiment, the biologically active agent is selected from the group consisting of leutinizing hormone releasing hormone (LHRH), LHRH analogs (such as goserelin, leuprolide, buserelin, triptorelin, gonadorelin, and napfarelin, menotropins (urofollitropin (FSH) and LH)), vasopressin, desmopressin, corticotropin (ACTH), ACTH analogs such as ACTH (1-24), calcitonin, vasopressin, deamino [Val4, D-Arg8] arginine vasopressin, interferon alpha, interferon beta, interferon gamma, erythropoietin (EPO), granulocyte macrophage colony stimulating factor (GM-CSF), granulocyte colony stimulating factor (G-CSF), interleukin-10 (IL-10), glucagon, growth hormone releasing factor (GHRF), insulin, insulinotropin, calcitonin, octreotide, endorphin, TRN, NT-36 (chemical name: N-[[(s)-4-oxo-2-azetidinyl]carbonyl]-L-histidyl-L-prolinamide), liprecin, aANF, bMSH, somatostatin, bradykinin, somatotropin, platelet-derived growth factor releasing factor, chymopapain, cholecystokinin, chorionic gonadotropin, epoprostenol (platelet aggregation inhibitor), glucagon, hirulog, interferons, interleukins, menotropins (urofollitropin (FSH) and LH), oxytocin, streptokinase, tissue plasminogen activator, urokinase, ANP, ANP clearance inhibitors, BNP, VEGF, angiotensin II antagonists, antidiuretic hormone agonists, bradykinn antagonists, ceredase, CSI's, calcitonin gene related peptide (CGRP), enkephalins, FAB fragments, IgE peptide suppressors, IGF-1, neurotrophic factors, colony stimulating factors, parathyroid hormone and agonists, parathyroid hormone antagonists, prostaglandin antagonists, pentigetide, protein C, protein S, renin inhibitors, thymosin alpha-1, thrombolytics, TNF, vasopressin antagonists analogs, alpha-1 antitrypsin (recombinant), TGF-beta, fondaparinux, ardeparin, dalteparin, defibrotide, enoxaparin, hirudin, nadroparin, reviparin, tinzaparin, pentosan polysulfate, oligonucleotides and oligonucleotide derivatives such as formivirsen, alendronic acid, clodronic acid, etidronic acid, ibandronic acid, incadronic acid, pamidronic acid, risedronic acid, tiludronic acid, zoledronic acid, argatroban, RWJ 445167, RWJ-671818, fentanyl, remifentanyl, sufentanyl, alfentanyl, lofentanyl, carfentanyl, and mixtures thereof.

In a further embodiment of the invention, the hydrogel formulation includes at least one pathway patency modulator or vasoconstrictor.

In another embodiment, the delivery system includes an applicator retainer that is adapted to cooperate with a pretreatment patch applicator, wherein the retainer includes a pretreatment patch seat adapted to receive the pretreatment patch and the backing membrane includes adhesive tabs adapted to adhere to the pretreatment patch seat. In a further embodiment, the pretreatment patch includes a supplemental adhesive ring disposed between the release liner ring and the skin adhesive ring that is adapted to cooperate with the skin adhesive ring.

Preferably, the retainer includes a pretreatment patch ring that is adapted to receive the pretreatment patch adhesive tabs during application of the pretreatment patch to the patient's skin, whereby the pretreatment patch is removable from the patient's skin by removing the retainer therefrom and whereby the skin template is disposed on the patient's skin.

In an alternative embodiment, the backing membrane includes a plurality of slots disposed proximate the periphery of the backing membrane and a plurality of break-away tabs adapted to cooperate with the pretreatment patch seat, and the retainer includes a pretreatment patch member having a plurality of posts that are adapted to engage the pretreatment patch slots during application of the pretreatment patch to the patient's skin, whereby the pretreatment patch is removable from the patient's skin by removing the retainer therefrom and whereby the skin template is disposed on the patient's skin.

In accordance with a further embodiment of the invention, the invention comprises a pretreatment member (or patch) for pre-treating a patient's skin having (i) a backing membrane and (ii) a microprojection array, the microprojection array being adhered to the backing membrane, the microprojection array including a plurality of microprojections adapted to pierce the stratum corneum of the patient, the pretreatment patch including a release liner ring that is removably secured to the backing membrane and a skin adhesive ring that is adhered to the release liner ring, the release liner ring and the skin adhesive ring being adapted to form a skin template on the patient's skin after the pretreatment patch is applied to and removed from the patient's skin.

In one embodiment of the invention, the pretreatment patch includes a polymeric membrane ring that is disposed between the release liner ring and the skin adhesive ring, wherein the skin template comprises the release liner ring, polymeric membrane ring and skin adhesive ring.

In a further embodiment, the pretreatment patch includes a polymeric membrane disposed between the backing membrane and the microprojection array.

Preferably, the microprojection array has a microprojection density in the range of 10-2000 microprojections/cm² and provides a treated skin area in the range of approximately 0.5-30 cm² after the pretreatment patch is applied to the skin of the patient.

The method for delivering a biologically active agent through the skin of a patient, in accordance with one embodiment of the invention, comprises the steps of (i) providing a pretreatment patch adapted to be placed on the patient's skin, the pretreatment patch having a backing membrane and a microprojection array, the microprojection array being adhered to the backing membrane, the microprojection array including a plurality of microprojections adapted to pierce the stratum corneum of the patient, the pretreatment patch including a release liner ring that is removably secured to the backing membrane and a skin adhesive ring that is adhered to the release liner ring, the release liner ring and the skin adhesive ring being adapted to form a skin template on the patient's skin after the pretreatment patch is applied to and removed from the patient's skin, (ii) providing a gel patch having a top and bottom surface, the gel patch including a reservoir containing a hydrogel formulation, the gel patch having a skin contact area in the range of approximately 0.5-30 cm², (iii) applying the pretreatment patch to the patient's skin, whereby the microprojections pierce the stratum corneum of the patient to provide a pretreated skin area having a plurality of microslits and whereby the skin template adheres to the patient's skin, (iv) removing the pretreatment patch from the patient's skin, and (v) applying the gel patch to the pretreated skin area, the gel patch being positioned over the skin template, whereby the hydrogel formulation is released from the reservoir and migrates into and through the microslits formed in the stratum corneum by the pretreatment patch.

Preferably, the gel patch includes a formulation membrane that is disposed proximate the gel patch reservoir that is adapted to inhibit migration of enzymes and/or bacteria into the hydrogel formulation.

In one embodiment of the invention, the pretreatment patch includes a polymeric membrane ring that is disposed between the release liner ring and the skin adhesive ring, wherein the skin template comprises the release liner ring, polymeric membrane ring and skin adhesive ring.

In another embodiment, the pretreatment patch includes a polymeric support membrane disposed between the backing membrane and the microprojection array.

Preferably, the microprojection array has a microprojection density in the range of 10-2000 microprojections/cm² and provides a pretreated skin area in the range of approximately 0.5-30 cm² after the pretreatment patch is applied to the skin of the patient.

In one embodiment of the invention, the pretreated skin area is substantially equal to the gel patch skin contact area. In a further embodiment, the pretreated skin area is greater than the gel patch skin contact area. In another embodiment, the pretreated skin area is smaller than the gel patch skin contact area.

Preferably, the hydrogel formulation comprises a water-based hydrogel. In one embodiment of the invention, the hydrogel formulation comprises a polymeric material and, optionally, a surfactant. In one aspect of the invention, the polymeric material comprises a cellulose derivative. In a further aspect of the invention, the polymeric material is selected from the group consisting of hydroxyethylcellulose (HEC), hydroxypropylmethylcellulose (HPMC), hydroxypropycellulose (HPC), methylcellulose (MC), hydroxyethylmethylcellulose (HEMC), ethylhydroxyethylcellulose (EHEC), carboxymethyl cellulose (CMC), poly(vinyl alcohol), poly(ethylene oxide), poly(2-hydroxyethylmethacrylate), poly(n-vinyl pyrolidone), pluronics, and mixtures thereof. In a further aspect of the invention, the surfactant is selected from the group consisting of Tween 20 and Tween 80.

In a preferred embodiment of the invention, the hydrogel formulation includes at least one biologically active agent, the biologically active agent being selected from the group consisting of small molecular weight compounds, polypeptides, proteins, oligonucleotides, nucleic acids and polysaccharides.

In an alternative embodiment, the biologically active agent is selected from the group consisting of leutinizing hormone releasing hormone (LHRH), LHRH analogs (such as goserelin, leuprolide, buserelin, triptorelin, gonadorelin, and napfarelin, menotropins (urofollitropin (FSH) and LH)), vasopressin, desmopressin, corticotropin (ACTH), ACTH analogs such as ACTH (1-24), calcitonin, vasopressin, deamino [Val4, D-Arg8] arginine vasopressin, interferon alpha, interferon beta, interferon gamma, erythropoietin (EPO), granulocyte macrophage colony stimulating factor (GM-CSF), granulocyte colony stimulating factor (G-CSF), interleukin-10 (IL-10), glucagon, growth hormone releasing factor (GHRF), insulin, insulinotropin, calcitonin, octreotide, endorphin, TRN, NT-36 (chemical name: N-[[(s)-4-oxo-2-azetidinyl]carbonyl]-L-histidyl-L-prolinamide), liprecin, aANF, bMSH, somatostatin, bradykinin, somatotropin, platelet-derived growth factor releasing factor, chymopapain, cholecystokinin, chorionic gonadotropin, epoprostenol (platelet aggregation inhibitor), glucagon, hirulog, interferons, interleukins, menotropins (urofollitropin (FSH) and LH), oxytocin, streptokinase, tissue plasminogen activator, urokinase, ANP, ANP clearance inhibitors, BNP, VEGF, angiotensin II antagonists, antidiuretic hormone agonists, bradykinn antagonists, ceredase, CSI's, calcitonin gene related peptide (CGRP), enkephalins, FAB fragments, IgE peptide suppressors, IGF-1, neurotrophic factors, colony stimulating factors, parathyroid hormone and agonists, parathyroid hormone antagonists, prostaglandin antagonists, pentigetide, protein C, protein S, renin inhibitors, thymosin alpha-1, thrombolytics, TNF, vasopressin antagonists analogs, alpha-1 antitrypsin (recombinant), TGF-beta, fondaparinux, ardeparin, dalteparin, defibrotide, enoxaparin, hirudin, nadroparin, reviparin, tinzaparin, pentosan polysulfate, oligonucleotides and oligonucleotide derivatives such as formivirsen, alendronic acid, clodronic acid, etidronic acid, ibandronic acid, incadronic acid, pamidronic acid, risedronic acid, tiludronic acid, zoledronic acid, argatroban, RWJ 445167, RWJ-671818, fentanyl, remifentanyl, sufentanyl, alfentanyl, lofentanyl, carfentanyl, and mixtures thereof.

In a preferred embodiment of the invention, the method includes the step of delivering up to 50 mg per day of the biologically active agent. Preferably, the noted delivery step comprises zero-order delivery.

In another embodiment of the invention, the hydrogel formulation includes at least one pathway patency modulator and/or vasoconstrictor.

In another embodiment, the method includes the step of providing an applicator retainer that is adapted to cooperate with a pretreatment patch applicator, wherein the retainer includes a pretreatment patch seat adapted to receive the pretreatment patch and the backing membrane includes adhesive tabs adapted to adhere to the pretreatment patch seat. In a further embodiment, the pretreatment patch includes a supplemental adhesive ring disposed between the release liner ring and the skin adhesive ring that is adapted to cooperate with the skin adhesive ring.

Preferably, the retainer includes a pretreatment patch ring that is adapted to receive the pretreatment patch adhesive tabs during the step of applying the pretreatment patch to the patient's skin, whereby the pretreatment patch is removable from the patient's skin by removing the retainer therefrom and whereby the skin template is disposed on the patient's skin.

In an alternative embodiment, the backing membrane includes a plurality of slots disposed proximate the periphery of the backing membrane and a plurality of break-away tabs adapted to cooperate with the pretreatment patch seat, and the retainer includes a pretreatment patch member having a plurality of posts that are adapted to engage the pretreatment patch slots during application of the pretreatment patch to the patient's skin, whereby the pretreatment patch is removable from the patient's skin by removing the retainer therefrom and whereby the skin template is disposed on the patient's skin.

### BRIEF DESCRIPTION OF THE DRAWINGS

Further features and advantages will become apparent from the following and more particular description of the preferred embodiments of the invention, as illustrated in the accompanying drawings, and in which like referenced characters generally refer to the same parts or elements throughout the views, and in which:

FIG. 1 is a perspective view of one embodiment of the gel patch, according to the invention;

FIG. 2 is a perspective view of one embodiment of the pretreatment patch, according to the invention;

FIG. 3 is a partial perspective view of one embodiment of a microprojection array, according to the invention;

FIG. 4 is a sectioned side plane view of one embodiment of a retainer having a pretreatment apparatus seated therein, according to the invention;

FIG. 5 is a perspective view of the retainer shown in FIG. 4;

FIG. 6 is a exploded diagrammatic view of one embodiment of the pretreatment and gel patches shown in FIGS. 1 and 2, according to the invention;

FIGS. 7 through 9 are exploded diagrammatic views of additional embodiments of the pretreatment patch shown in FIG. 2, according to the invention;

FIG. 10 is a side plane view of one embodiment of the assembled drug delivery system, according to the invention;

FIG. 11 is a schematic illustration of the placement of the gel patch on one embodiment of the skin template, according to the invention;

FIG. 12 is a diagrammatic view of a further embodiment of a skin template, according to the invention;

FIG. 13 is schematic illustration of a gel patch placed on the skin of a patient, according to the invention.

FIGS. 14 and 15 are exploded diagrammatic views of further embodiments of the pretreatment patch shown in FIG. 1, according to the invention;

FIG. 16 is a sectioned side plane view of a further embodiment of a retainer having a pretreatment patch seated therein, according to the invention;

FIG. 17 is a top plane view of a further embodiment of a pretreatment device having extending break-away tabs, according to the invention;

FIG. 18 is a sectioned side plane view of another embodiment of a retainer having the pretreatment patch shown in FIG. 17 seated therein, according to the invention;

FIG. 19 is a graph showing the time dependent flux of pentosan polysulfate (PPS) through the skin of a living hairless guinea pig employing one embodiment of the drug delivery system of the present invention;

FIGS. 20 and 21 are graphs showing the concentration dependant flux of an oligonucleotide through the skin of a living hairless guinea pig, employing one embodiment of the drug delivery system of the present invention; and

FIGS. 22 and 23 are further graphs showing the concentration dependant flux of an oligonucleotide through the skin of a living hairless guinea pig.

### DETAILED DESCRIPTION OF THE INVENTION

Before describing the present invention in detail, it is to be understood that this invention is not limited to particularly exemplified materials, methods or structures as such may, of course, vary. Thus, although a number of materials and methods similar or equivalent to those described herein can be used in the practice of the present invention, the preferred materials and methods are described herein.

It is also to be understood that the terminology used herein is for the purpose of describing particular embodiments of the invention only and is not intended to be limiting.

Unless defined otherwise, all technical and scientific terms used herein have the same meaning as commonly understood by one having ordinary skill in the art to which the invention pertains.

Further, all publications, patents and patent applications cited herein, whether supra or infra, are hereby incorporated by reference in their entirety.

Finally, as used in this specification and the appended claims, the singular forms “a, “an” and “the” include plural referents unless the content clearly dictates otherwise. Thus, for example, reference to “an active agent” includes two or more such agents; reference to “a microprojection” includes two or more such microprojections and the like.

### Definitions

The term “transdermal”, as used herein, means the delivery of an agent into and/or through the skin for local or systemic therapy.

The term “transdermal flux”, as used herein, means the rate of transdermal delivery.

The term “co-delivering”, as used herein, means that a supplemental agent(s) is administered transdermally either before the agent is delivered, before and during transdermal flux of the agent, during transdermal flux of the agent, during and after transdermal flux of the agent, and/or after transdermal flux of the agent. Additionally, two or more biologically active agents may be formulated in the hydrogel formulation, resulting in co-delivery of the biologically active agents.

The term “biologically active agent”, as used herein, refers to a composition of matter or mixture containing a drug which is pharmacologically effective when administered in a therapeutically effective amount. Examples of such active agents include, without limitation, small molecular weight compounds, polypeptides, proteins, oligonucleotides, nucleic acids and polysaccharides.

Further examples of “biologically active agents” include, without limitation, leutinizing hormone releasing hormone (LHRH), LHRH analogs (such as goserelin, leuprolide, buserelin, triptorelin, gonadorelin, and napfarelin, menotropins (urofollitropin (FSH) and LH)), vasopressin, desmopressin, corticotropin (ACTH), ACTH analogs such as ACTH (1-24), calcitonin, vasopressin, deamino [Val4, D-Arg8] arginine vasopressin, interferon alpha, interferon beta, interferon gamma, erythropoietin (EPO), granulocyte macrophage colony stimulating factor (GM-CSF), granulocyte colony stimulating factor (G-CSF), interleukin-10 (IL-10), glucagon, growth hormone releasing factor (GHRF), insulin, insulinotropin, calcitonin, octreotide, endorphin, TRN, NT-36 (chemical name: N-[[(s)-4-oxo-2-azetidinyl]carbonyl]-L-histidyl-L-prolinamide), liprecin, aANF, bMSH, somatostatin, bradykinin, somatotropin, platelet-derived growth factor releasing factor, chymopapain, cholecystokinin, chorionic gonadotropin, epoprostenol (platelet aggregation inhibitor), glucagon, hirulog, interferons, interleukins, menotropins (urofollitropin (FSH) and LH), oxytocin, streptokinase, tissue plasminogen activator, urokinase, ANP, ANP clearance inhibitors, BNP, VEGF, angiotensin II antagonists, antidiuretic hormone agonists, bradykinn antagonists, ceredase, CSI's, calcitonin gene related peptide (CGRP), enkephalins, FAB fragments, IgE peptide suppressors, IGF-1, neurotrophic factors, colony stimulating factors, parathyroid hormone and agonists, parathyroid hormone antagonists, prostaglandin antagonists, pentigetide, protein C, protein S, renin inhibitors, thymosin alpha-1, thrombolytics, TNF, vasopressin antagonists analogs, alpha-1 antitrypsin (recombinant), TGF-beta, fondaparinux, ardeparin, dalteparin, defibrotide, enoxaparin, hirudin, nadroparin, reviparin, tinzaparin, pentosan polysulfate, oligonucleotides and oligonucleotide derivatives such as formivirsen, alendronic acid, clodronic acid, etidronic acid, ibandronic acid, incadronic acid, pamidronic acid, risedronic acid, tiludronic acid, zoledronic acid, argatroban, RWJ 445167, and RWJ-671818, fentanyl, remifentanyl, sufentanyl, alfentanyl, lofentanyl, carfentanyl, and mixtures thereof.

The noted biologically active agents can also be in various forms, such as free bases, acids, charged or uncharged molecules, components of molecular complexes or nonirritating, pharmacologically acceptable salts. Further, simple derivatives of the active agents (such as ethers, esters, amides, etc.), which are easily hydrolyzed at body pH, enzymes, etc., can be employed.

The term “biologically active agent”, as used herein, also refers to a composition of matter or mixture containing a “vaccine” or other immunologically active agent or an agent which is capable of triggering the production of an immunologically active agent, and which is directly or indirectly immunologically effective when administered in an immunologically effective amount.

The term “vaccine”, as used herein, refers to conventional and/or commercially available vaccines, including, but not limited to, flu vaccines, Lyme disease vaccine, rabies vaccine, measles vaccine, mumps vaccine, chicken pox vaccine, small pox vaccine, hepatitis vaccine, pertussis vaccine, and diphtheria vaccine, recombinant protein vaccines, DNA vaccines and therapeutic cancer vaccines. The term “vaccine” thus includes, without limitation, antigens in the form of proteins, polysaccharides, oligosaccharides, lipoproteins, weakened or killed viruses such as cytomegalovirus, hepatitis B virus, hepatitis C virus, human papillomavirus, rubella virus, and varicella zoster, weakened or killed bacteria such as bordetella pertussis, clostridium tetani, corynebacterium diphtheriae, group A streptococcus, legionella pneumophila, neisseria meningitides, pseudomonas aeruginosa, streptococcus pneumoniae, treponema pallidum, and vibrio cholerae and mixtures thereof.

It is to be understood that more than one biologically active agent may be incorporated into the hydrogel formulations of this invention, and that the use of the term “biologically active agent” (or “active agent”) in no way excludes the use of two or more such active agents.

The term “biologically effective amount” or “biologically effective rate” shall be used when the biologically active agent is a pharmaceutically active agent and refers to the amount or rate of the pharmacologically active agent needed to effect the desired therapeutic, often beneficial, result. The amount of active agent employed in the hydrogel formulations of the invention will be that amount necessary to deliver a therapeutically effective amount of the active agent to achieve the desired therapeutic result. In practice, this will vary widely depending upon the particular pharmacologically active agent being delivered, the site of delivery, the severity of the condition being treated, the desired therapeutic effect and the release kinetics for delivery of the agent from the hydrogel into skin tissues.

The term “biologically effective amount” or “biologically effective rate” shall also be used when the biologically active agent is an immunologically active agent and refers to the amount or rate of the immunologically active agent needed to stimulate or initiate the desired immunologic, often beneficial result. The amount of the immunologically active agent employed in the hydrogel formulations of the invention will be that amount necessary to deliver an amount of the active agent needed to achieve the desired immunological result. In practice, this will vary widely depending upon the particular immunologically active agent being delivered, the site of delivery, and the dissolution and release kinetics for delivery of the active agent into skin tissues.

The term “vasoconstrictor”, as used herein, refers to a composition of matter or mixture that narrows the lumen of blood vessels and, hence, reduces peripheral blood flow. Examples of suitable vasoconstrictors include, without limitation, amidephrine, cafaminol, cyclopentamine, deoxyepinephrine, epinephrine, felypressin, indanazoline, metizoline, midodrine, naphazoline, nordefrin, octodrine, ornipressin, oxymethazoline, phenylephrine, phenylethanolamine, phenylpropanolamine, propylhexedrine, pseudoephedrine, tetrahydrozoline, tramazoline, tuaminoheptane, tymazoline, vasopressin, xylometazoline and the mixtures thereof.

The term “microprojections”, as used herein, refers to piercing elements which are adapted to pierce or cut through the stratum corneum into the underlying epidermis layer, or epidermis and dermis layers, of the skin of a living animal, particularly a mammal and more particularly a human.

As discussed in detail herein, in one embodiment of the invention, the microprojections preferably have a projection length of less than 500 microns, more preferably, less than 250 microns. The microprojections typically have a width and thickness of about 5 to 50 microns. The microprojections may be formed in different shapes, such as needles, blades, pins, punches, and combinations thereof.

The term “microprojection array”, as used herein, refers to a plurality of microprojections arranged in an array for piercing the stratum corneum. The microprojection array may be formed by etching or punching a plurality of microprojections from a thin sheet and folding or bending the microprojections out of the plane of the sheet to form a configuration, such as that shown in FIG. 3. The microprojection array may also be formed in other known manners, such as by forming one or more strips having microprojections along an edge of each of the strip(s) as disclosed in U.S. Pat. No. 6,050,988, which is incorporated by reference herein in its entirety.

As indicated above, the present invention comprises a pretreatment method and system for enhancing transdermal delivery of a biologically active agent (i.e., drug, active, etc.) to a patient. The pretreatment or delivery system generally includes a pretreatment patch having a plurality of stratum corneum-piercing microprojections extending therefrom and a gel patch having a hydrogel formulation that contains at least one biologically active agent. As will be readily appreciated by on having ordinary skill in the art, the delivery system facilitates transdermal “zero-order” delivery of up to 50 mg of a biologically active agent for up to approximately 24 hours (i.e., one day).

As will be appreciated by one having ordinary skill in the art, the present invention has utility in connection with the delivery of biologically active agents within any of the broad class of drugs normally delivered though body surfaces and membranes, including skin. In general, this includes drugs in all of the major therapeutic areas.

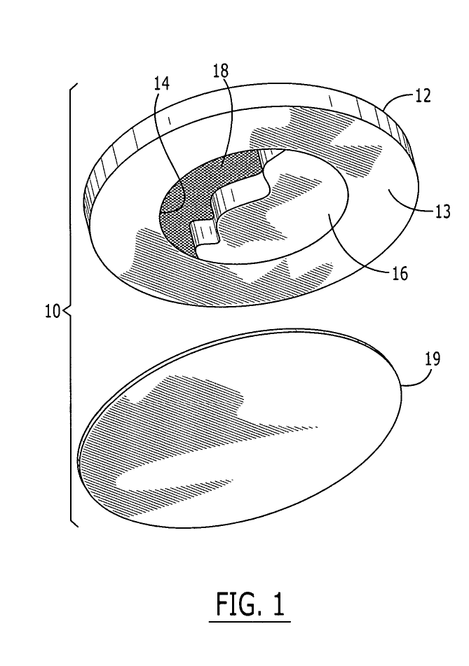

Referring now to FIG. 1, there is shown one embodiment of the gel patch 10. As illustrated in FIG. 1, the gel patch 10 includes a housing or ring 12 having a centrally disposed reservoir or opening 14 that is adapted to receive a predetermined amount of a hydrogel formulation 16 therein. The term “ring”, as used herein, is not limited to circular or oval shapes, but also includes polygonal shapes and/or polygonal shapes with rounded edges. The gel patch 10 further includes a backing member 18 that is disposed on the top surface of the ring 12 and a release liner 19 that is disposed on the bottom surface 13 of the ring 12. Preferably, the backing member 18 is impermeable to the hydrogel formulation.

According to the invention, the gel patch 10 has a skin contact area, which is defined by the opening 14, in the range of approximately 0.5-30 cm². More preferably, the skin contact area is in the range of approximately 1-10 cm². Even more preferably, the skin contact area is approximately 2 cm².

As illustrated in FIG. 1, the “total” skin contact area, which is defined as the area of the ring 12 or backing member 18, is generally larger than the noted skin contact area. According to the invention, the total skin contact area can be in the range of 1-60 cm².

In a further embodiment of the invention, the gel patch 10 includes a formulation membrane (not shown) that is disposed between the hydrogel formulation 16 and release liner 19. According to the invention, the formulation membrane has a pore size greater than the size of the biologically active agent contained in the hydrogel formulation 16 to avoid enzymatic and/or bacterial leakage into the formulation after removal of the liner 19 and placement of the gel patch 10 on the patient's skin. The formulation membrane is preferably a dialysis membrane.

Preferably, the ring 12 is constructed out of a resilient polymeric material, such as PETG (polyethylene terephthalate, Glycol modified), polyethylene, or polyurethane. In a preferred embodiment, the ring 12 is constructed of closed or open-cell foam. The foam preferably, but not exclusively, comprises polyethylene, polyurethane, neoprene, natural rubber, SBR, butyl, butadiene, nitrile, EPDM, ECH, polystyrene, polyester, polyether, polypropylene, EVA, EMA, metallocene resin, PVC, and blends of the above.

According to the invention, the gel patch 10 has a correspondingly similar shape and planar dimension (e.g., diameter) as the pretreatment patch (e.g., 20a). More preferably, the skin contact area of the gel patch 10 is substantially similar to the skin area pretreated by the pretreatment patch 20 (i.e., pretreated or effected area). In alternative embodiments of the invention, the skin contact area is slightly larger or smaller than the pretreated area.

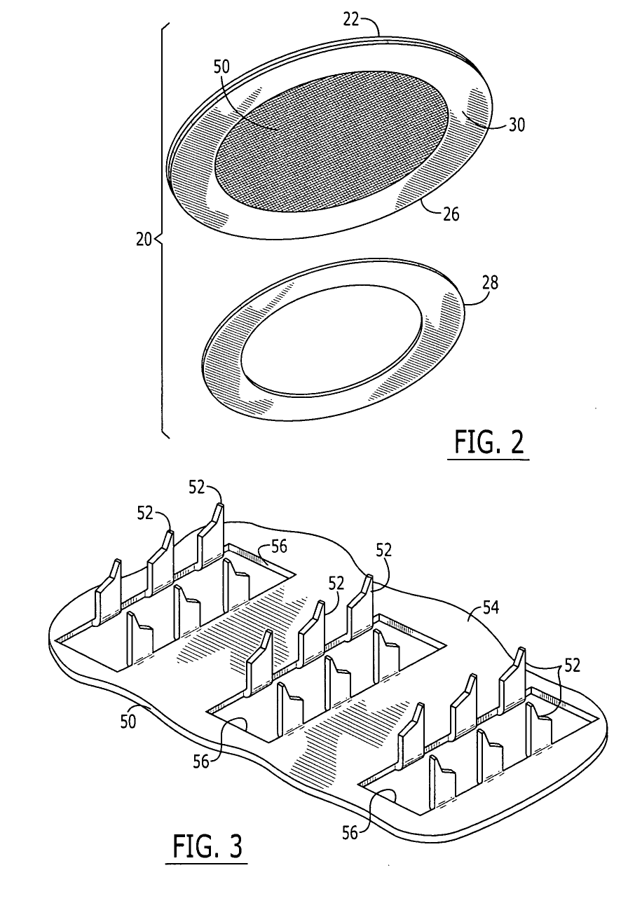

Referring now to FIG. 2, there is shown one embodiment of the pretreatment patch 20. As illustrated in FIG. 2, the pretreatment patch 20 includes a backing membrane 22 and a microprojection array 50. The pretreatment patch 20 further includes a release liner ring 26 and a skin adhesive ring 28 that is disposed on the non-release liner side 30 of the release liner ring 26.

Preferably, the backing membrane 22 is constructed out of a polymeric material, such as polyethylene, polyurethane or polypropylene. In a preferred embodiment, the backing membrane is constructed out of a polyethylene medical tape.

Preferably, the release liner ring 26 comprises a polyester film having a silicon release agent disposed on the release side of the ring 26. In a preferred embodiment of the invention, the release liner ring 26 has a thickness in the range of approximately 25-150 microns, more preferably, in the range of approximately 50-100 microns, even more preferably, approximately 75 microns.

Preferably, the polymeric membrane ring 34 comprises a polyester film. In a preferred embodiment of the invention, the polymeric membrane ring 34 has a thickness in the range of approximately 25-150 microns, more preferably, in the range of approximately 50-100 microns, even more preferably, approximately 75 microns.

Referring now to FIG. 3, there is shown one embodiment of the microprojection array 50. As illustrated in FIG. 3, the microprojection array 50 includes a plurality of microprojections 52 that extend downward from one surface of a sheet or plate 54.

The microprojections 52 are preferably sized and shaped to penetrate the stratum corneum of the epidermis when pressure is applied to the pretreatment patch 20. The microprojections 52 are further adapted to form microslits in the stratum corneum (i.e., pretreated area) to enhance the transdermal flux of the hydrogel formulation and, hence, biologically active agent contained therein, through the stratum corneum to achieve local or systemic therapy.

The microprojections 52 are generally formed from a single piece of sheet material and are sufficiently sharp and long to puncture the stratum corneum of the skin. In the illustrated embodiment, the sheet 54 is formed with an opening 56 between the microprojections 52. However, according to the invention, the microprojection array 50 need not include openings 56 or any retention features. Thus, in one embodiment of the invention, the microprojection array 50 does not include openings or retainer projections.

Preferably, the microprojections 52 have a projection length less than approximately 500 microns. In one embodiment, the microprojections have a projection length less than 250 microns.

According to the invention, the number of microprojections 52 in the microprojection array 50 is variable with respect to the desired flux rate, agent being sampled or delivered, delivery or sampling device used (i.e., electrotransport, passive, osmotic, pressure-driven, etc.), and other factors as will be evident to one of ordinary skill in the art. In general, the larger the number of microprojections per unit area (i.e., microprojection density), the more distributed is the flux of the agent through the skin because there are more pathways.

Preferably, the microprojection density is at least approximately 10 microprojections/cm². In one embodiment of the invention, the microprojection density is in the range of approximately 200-1000 microprojections/cm².

Further details of microprojection array 50 described above and other microprojection devices and arrays that can be employed within the scope of the invention are disclosed in U.S. Pat. No. 6,322,808, U.S. Pat. No. 6,230,051 B1 and Co-Pending U.S. application Ser. No. 10/045,842, which are incorporated by reference herein in their entirety.

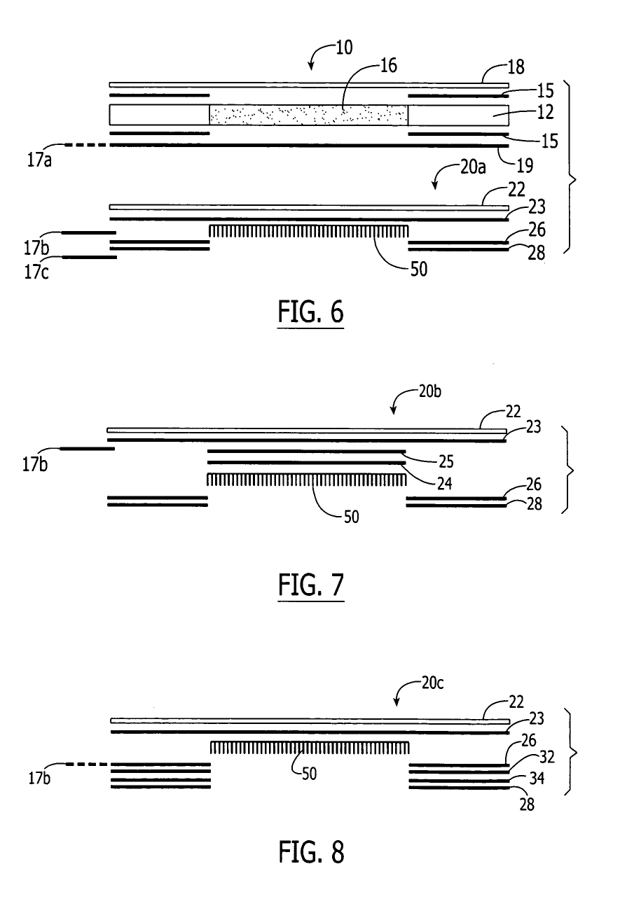

Referring now to FIG. 6, the assembly of one embodiment of the gel patch 10 and pretreatment patch, designated generally 20a, will be described in detail. Referring first to the gel patch 10, the backing member 18 is adhered to the top surface of the ring 12 via a conventional adhesive ring 15. A strippable release liner 19 is similarly adhered to the bottom surface of the gel patch ring 12 via a conventional adhesive ring 15. As described in detail below, the release liner 19 is removed prior to application of the gel patch 10 to the skin surface (or skin template 7, described in detail below).

Referring now to the pretreatment patch 20a, the backing membrane 22 is adhered to the microprojection array 50 via a conventional adhesive 23. According to the invention, the release liner side of the release liner ring 26 is adhered to the adhesive layer 23. The skin adhesive ring 28 is similarly adhered to the non-release liner side 30 of the release liner ring 26.

Optionally, the gel patch 10 and pretreatment patch 20a can include release tabs 17a, 17b and 17c. According to the invention, the tabs 17a, 17b, 17c can be formed integrally with the release liners (e.g., release liner 19) or be disposed between the liner(s) (e.g., release liner ring 26) and the adhesive layer 23. The tabs 17a, 17b, 17c can also be superposed, numbered or color-coded for the convenience of the user.

Referring now to FIG. 7, there is shown a further embodiment of the pretreatment patch, designated generally 20b. In the noted embodiment, the pretreatment patch 20b includes a polymeric membrane 25 that is adhered to the backing membrane 22 through the adhesive layer 23. The polymeric membrane 25 is also adhered to the microprojection array 50 by an adhesive layer 24.

According to the invention, the polymeric membrane 25 has a thickness substantially similar to the thickness of the release liner ring 26 discussed above. In a preferred embodiment of the invention, the polymeric membrane 25 comprises a polyester film.

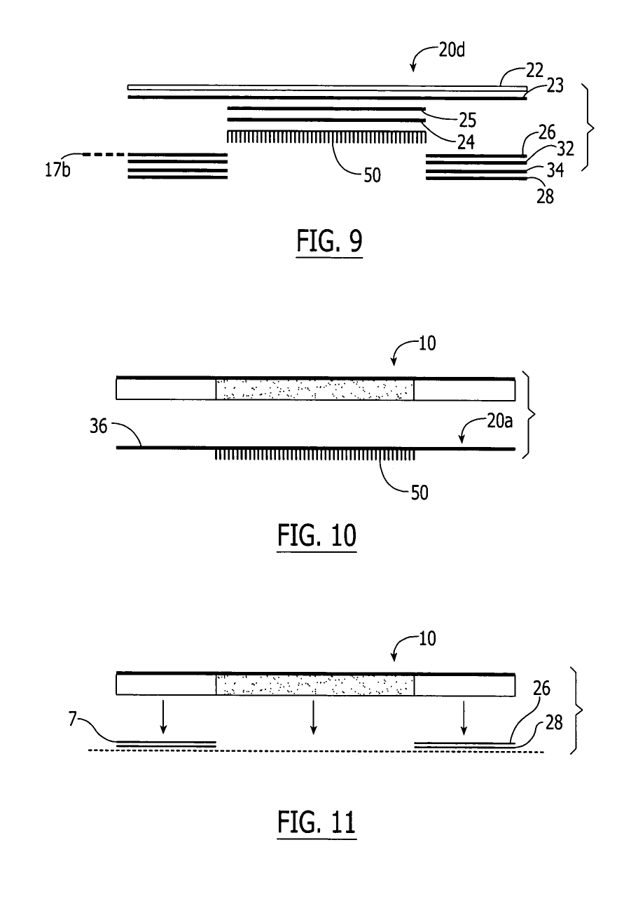

Referring now to FIG. 8, in a further embodiment of the invention, the pretreatment patch, designated generally 20c, includes a polymeric membrane ring 34 that is disposed between the skin adhesive ring 28 and an adhesive ring 32. Additionally, the non-release liner side of the release liner ring 26 is adhered to the adhesive layer 23, and the release liner side of the release liner ring 26 is adhered to the adhesive layer 32. In an alternative embodiment, the pretreatment patch, designated generally 20d, can also include the polymeric membrane 25 shown in FIG. 7 (see FIG. 9).

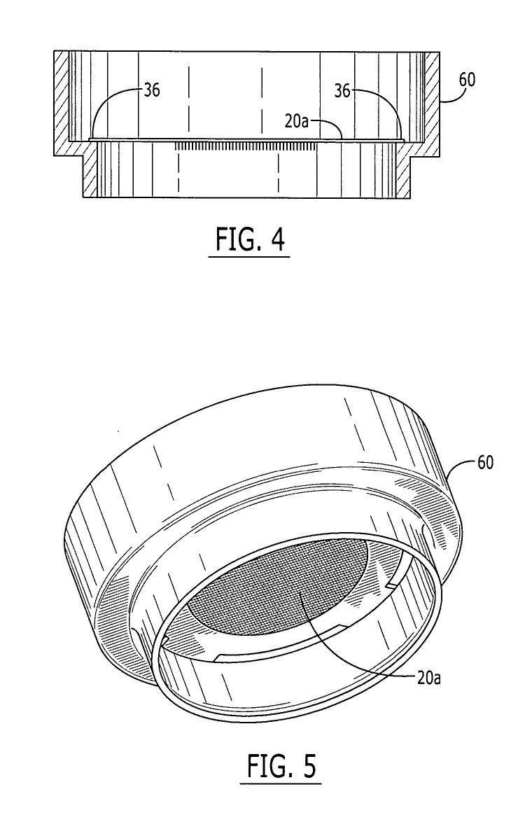

For storage and application, the pretreatment patch 20a (or 20b, 20c or 20d) is preferably suspended in a retainer ring 60 by adhesive tabs 36, as illustrated in FIG. 4 and described in detail in Co-Pending U.S. application Ser. No. 09/976,762 (Pub. No. 2002/0091357), which is incorporated by reference herein in its entirety.

Referring now to FIGS. 10-13, the preferred mode of employing one embodiment of the drug delivery system will be described in detail. Referring first to FIG. 10, the pretreatment patch (i.e., 20a, 20b, 20c or 20d) is applied to the patient's skin preferably using an impact applicator, such as the applicator disclosed in U.S. application Ser. No. 09/976,798 (Pub. No. 2002/0123675), which is incorporated by reference herein in its entirety.

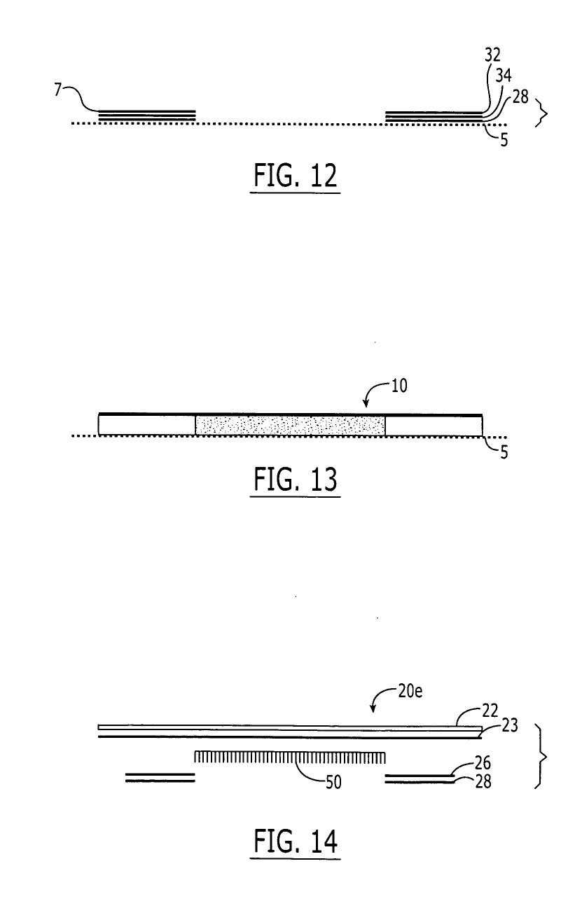

Immediately following application, the pretreatment patch, e.g., 20a, is removed from the patient's skin (optionally, by peeling the patch 20a via tab 17b) and discarded, leaving a “skin template” (denoted generally 7) comprising (i) the skin adhesive ring 28 and release liner ring 26 adhered to the skin surface 5 (see FIG. 11) or (ii) the skin adhesive ring 28, the polymeric membrane ring 34 and adhesive ring 32 adhered to the skin surface (see FIG. 12).

The release liner 19 of the gel patch 10 is then removed and the gel patch 10 is placed on the template 7 (as shown in FIG. 13), whereby the hydrogel formulation 16 is released from the gel patch 10 and passes through the microslits in the stratum corneum formed by the pretreatment patch 20a.

In a further embodiment of the invention, the pretreatment patch, designated generally 20e, comprises the configuration shown in FIG. 14, which is similar to the configuration shown in FIG. 6, and is adapted to seat in the retainer 62 shown in FIG. 16. As illustrated in FIG. 16, the retainer 62 preferably includes an internal ring or ridge 63 proximate the bottom portion of the retainer 62.

According to the invention, during application of the pretreatment patch 20e, the adhesive layer 23 adheres to the ring 63. The pretreatment patch 20e can then be readily removed from the skin by lifting off the applicator/retainer ring assembly, leaving the skin template 7, comprising the skin adhesive ring 28 and release liner ring 26.

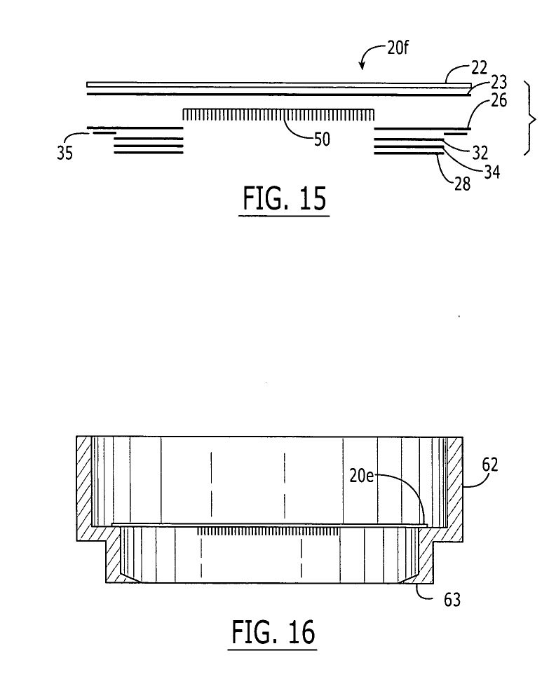

Referring now to FIG. 15, in an alternative embodiment of the invention, the pretreatment patch, designated generally 20f, includes an additional adhesive ring 35 that ensures adhesion of the pretreatment patch 20 to the retainer ring 63 during the application process.

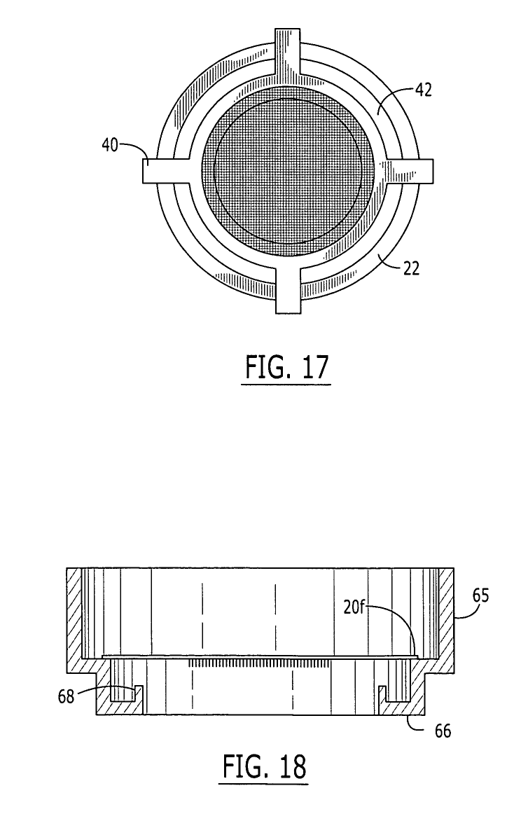

Referring now to FIG. 17, in yet another embodiment of the invention, the pretreatment patch 20f includes a plurality of slots 42 that extend through components and/or layers 22, 23, 26, 32, 34, 28 and, if employed, 35 and a plurality of tabs 40 that extend from the ring 22. The pretreatment patch 20f is adapted to seat in the retainer 65 shown in FIG. 18.

As illustrated in FIG. 18, the retainer 65 includes a plurality of posts 68 that are disposed on the retainer ring 66. According to the invention, during application of the pretreatment patch 20f, the tabs 40 break off and release the patch 20f. The posts 68 are then received by the slots 42 on the pretreatment patch ring 22. The pretreatment patch 20f can then similarly be removed from the skin by lifting off the applicator/retainer ring assembly.

After application of the noted pretreatment patches 20e, 20f, the release liner 19 of the gel patch 10 is similarly removed and the gel patch 10 is placed on the template 7, whereby the hydrogel formulation 16 is released from the gel patch 10 and passes through the microslits in the stratum corneum formed by the pretreatment patch 20e or 20f.

Preferably, the hydrogel formulation of the invention comprises water-based hydrogels, such as the hydrogel formulations disclosed in Co-Pending application Ser. No. 60/514,433, which is incorporated by reference herein in its entirety.

As is well known in the art, hydrogels are macromolecular polymeric networks that are swollen in water. Examples of suitable polymeric networks include, without limitation, hydroxyethylcellulose (HEC), hydroxypropylmethylcellulose (HPMC), hydroxypropycellulose (HPC), methylcellulose (MC), hydroxyethylmethylcellulose (HEMC), ethylhydroxyethylcellulose (EHEC), carboxymethyl cellulose (CMC), poly(vinyl alcohol), poly(ethylene oxide), poly(2-hydroxyethylmethacrylate), poly(n-vinyl pyrolidone), and pluronics. The most preferred polymeric materials are cellulose derivatives. These polymers can be obtained in various grades presenting different average molecular weight and therefore exhibit different rheological properties.

According to the invention, the hydrogel formulations contain at least one biologically active agent. Preferably, the biologically active agent comprises one of the aforementioned active agents, including, without limitation, leutinizing hormone releasing hormone (LHRH), LHRH analogs (such as goserelin, leuprolide, buserelin, triptorelin, gonadorelin, and napfarelin, menotropins (urofollitropin (FSH) and LH)), vasopressin, desmopressin, corticotropin (ACTH), ACTH analogs such as ACTH (1-24), calcitonin, vasopressin, deamino [Val4, D-Arg8] arginine vasopressin, interferon alpha, interferon beta, interferon gamma, erythropoietin (EPO), granulocyte macrophage colony stimulating factor (GM-CSF), granulocyte colony stimulating factor (G-CSF), interleukin-10 (IL-10), glucagon, growth hormone releasing factor (GHRF), insulin, insulinotropin, calcitonin, octreotide, endorphin, TRN, NT-36 (chemical name: N-[[(s)-4-oxo-2-azetidinyl]carbonyl]-L-histidyl-L-prolinamide), liprecin, aANF, bMSH, somatostatin, bradykinin, somatotropin, platelet-derived growth factor releasing factor, chymopapain, cholecystokinin, chorionic gonadotropin, epoprostenol (platelet aggregation inhibitor), glucagon, hirulog, interferons, interleukins, menotropins (urofollitropin (FSH) and LH), oxytocin, streptokinase, tissue plasminogen activator, urokinase, ANP, ANP clearance inhibitors, BNP, VEGF, angiotensin II antagonists, antidiuretic hormone agonists, bradykinn antagonists, ceredase, CSI's, calcitonin gene related peptide (CGRP), enkephalins, FAB fragments, IgE peptide suppressors, IGF-1, neurotrophic factors, colony stimulating factors, parathyroid hormone and agonists, parathyroid hormone antagonists, prostaglandin antagonists, pentigetide, protein C, protein S, renin inhibitors, thymosin alpha-1, thrombolytics, TNF, vasopressin antagonists analogs, alpha-1 antitrypsin (recombinant), TGF-beta, fondaparinux, ardeparin, dalteparin, defibrotide, enoxaparin, hirudin, nadroparin, reviparin, tinzaparin, pentosan polysulfate, oligonucleotides and oligonucleotide derivatives such as formivirsen, alendronic acid, clodronic acid, etidronic acid, ibandronic acid, incadronic acid, pamidronic acid, risedronic acid, tiludronic acid, zoledronic acid, argatroban, RWJ 445167, RWJ-671818, fentanyl, remifentanyl, sufentanyl, alfentanyl, lofentanyl and carfentanyl.

More preferably, the biologically active agent comprises a biologically active agent selected from the group consisting of small molecular weight compounds, polypeptides, proteins, oligonucleotides, nucleic acids and polysaccharides.

Even more preferably, the biologically active agent comprises a pharmacological agent requiring a daily dose of less than 50 mg per day. The noted pharmacological agent further preferably has a solubility greater than 10 mg/mL in the hydrogel formulation.

According to the invention, the hydrogel formulations also include one surfactant (i.e., wetting agent). According to the invention, the surfactant(s) can be zwitterionic, amphoteric, cationic, anionic, or nonionic. Examples of surfactants include, sodium lauroamphoacetate, sodium dodecyl sulfate (SDS), cetylpyridinium chloride (CPC), dodecyltrimethyl ammonium chloride (TMAC), benzalkonium, chloride, polysorbates, such as Tween 20 and Tween 80, other sorbitan derivatives such as sorbitan laurate, and alkoxylated alcohols such as laureth-4. Most preferred surfactants include Tween 20, Tween 80, and SDS.

Preferably, the hydrogel formulations further include polymeric materials or polymers having amphiphilic properties. Examples of the noted polymers include, without limitation, cellulose derivatives, such as hydroxyethylcellulose (HEC), hydroxypropylmethylcellulose (HPMC), hydroxypropycellulose (HPC), methylcellulose (MC), hydroxyethylmethylcellulose (HEMC), or ethylhydroxyethylcellulose (EHEC), as well as pluronics.

Preferably, the concentration of the surfactant is comprised between 0.001% and 2 wt. % of the hydrogel formulation. The concentration of the polymer that exhibits amphiphilic properties is preferably in the range of approximately 0.5-40 wt. % of the hydrogel formulation.

In a preferred embodiment, the hydrogel formulations of the invention contain at least one pathway patency modulator or “anti-healing agent”, such as those disclosed in Co-Pending U.S. application Ser. No. 09/950,436, which is incorporated by reference herein in its entirety. As set forth in the noted Co-Pending Application, the anti-healing agents prevent or diminish the skin's natural healing processes thereby preventing the closure of the pathways or microslits formed in the stratum corneum by the microprojection member 20. Examples of anti-healing agents include, without limitation, osmotic agents (e.g., sodium chloride), and zwitterionic compounds (e.g., amino acids).

The term “anti-healing agent”, as defined in the Co-Pending Application, further includes anti-inflammatory agents, such as betamethasone 21-phosphate disodium salt, triamcinolone acetonide 21-disodium phosphate, hydrocortamate hydrochloride, hydrocortisone 21-phosphate disodium salt, methylprednisolone 21-phosphate disodium salt, methylprednisolone 21-succinaate sodium salt, paramethasone disodium phosphate and prednisolone 21-succinate sodium salt, and anticoagulants, such as citric acid, citrate salts (e.g., sodium citrate), dextrin sulfate sodium, and EDTA.

According to the invention, the hydrogel formulations can also include anon-aqueous solvent, such as ethanol, isopropanol, propylene glycol, polyethylene glycol and the like, dyes, pigments, inert fillers, permeation enhancers, excipients, and other conventional components of pharmaceutical products or transdermal devices known in the art.

The hydrogel formulations can further include at least one vasoconstrictor. Suitable vasoconstrictors include, without limitation, epinephrine, naphazoline, tetrahydrozoline indanazoline, metizoline, tramazoline, tymazoline, oxymetazoline, xylometazoline, amidephrine, cafaminol, cyclopentamine, deoxyepinephrine, epinephrine, felypressin, indanazoline, metizoline, midodrine, naphazoline, nordefrin, octodrine, ornipressin, oxymethazoline, phenylephrine, phenylethanolamine, phenylpropanolamine, propylhexedrine, pseudoephedrine, tetrahydrozoline, tramazoline, tuaminoheptane, tymazoline, vasopressin and xylometazoline, and the mixtures thereof.

It will be appreciated by one having ordinary skill in the art that in order to facilitate drug transport across the skin barrier, the present invention can also be employed in conjunction with a wide variety of iontophoresis or electrotransport systems, as the invention is not limited in any way in this regard. Illustrative electrotransport drug delivery systems are disclosed in U.S. Pat. No. 5,147,296, U.S. Pat. No. 5,080,646, U.S. Pat. No. 5,169,382 and U.S. Pat. No. 5,169383, the disclosures of which are incorporated by reference herein in their entirety.

The term “electrotransport” refers, in general, to the passage of a beneficial agent, e.g., a drug or drug precursor, through a body surface such as skin, mucous membranes, nails, and the like. The transport of the agent is induced or enhanced by the application of an electrical potential, which results in the application of electric current, which delivers or enhances delivery of the agent, or, for “reverse” electrotransport, samples or enhances sampling of the agent. The electrotransport of the agents into or out of the human body may by attained in various manners.

One widely used electrotransport process, iontophoresis, involves the electrically induced transport of charged ions. Electroosmosis, another type of electrotransport process involved in the transdermal transport of uncharged or neutrally charged molecules (e.g., transdermal sampling of glucose), involves the movement of a solvent with the agent through a membrane under the influence of an electric field. Electroporation, still another type of electrotransport, involves the passage of an agent through pores formed by applying an electrical pulse, a high voltage pulse, to a membrane.

In many instances, more than one of the noted processes may be occurring simultaneously to different extents. Accordingly, the term “electrotransport” is given herein its broadest possible interpretation, to include the electrically induced or enhanced transport of at least one charged or uncharged agent, or mixtures thereof, regardless of the specific mechanism(s) by which the agent is actually being transported. Additionally, other transport enhancing methods such as sonophoresis or piezoelectric devices can be used in conjunction with the invention.

When the invention is employed in conjunction with electrotransport, sonophoresis, or piezoelectric systems, the microprojection member 20 is first applied to the skin as explained above. The release liner 19 is removed from the gel pack 10, which is part of an electrotransport, sonophoresis, or piezoelectric system. This assembly is then placed on the skin template 7, whereby the hydrogel formulation 16 is released from the gel patch 10 and passes through the microslits in the stratum corneum formed by the pretreatment patch 20a, 20b, 20c, or 20d to achieve local or systemic therapy with additional facilitation of drug transport provided by electrotransport, sonophoresis, or piezoelectric processes. When the invention is employed in conjunction with electrotransport, sonophoresis, or piezoelectric systems, the total skin contact area can be in the range of 2-120 cm².

### EXAMPLES

The following examples are given to enable those skilled in the art to more clearly understand and practice the present invention. They should not be considered as limiting the scope of the invention but merely as being illustrated as representative thereof.

### Example 1

As is well known in the art, pentosan polysulfate (PPS) is a highly negatively charged compound that typically does not penetrate the skin significantly without the use of penetration enhancers or physical disruption of the skin barrier. In this experiment, PPS was delivered by passive diffusion through skin pathways created by a pretreatment device having a microprojection array. The microprojection array comprised a stainless steel sheet having a thickness of 0.025 mm, trapezoidally shaped microprojections bent at an angle of approximately 90° to the plane of the sheet and a microprojection density of 241 microprojections/cm². The microprojections had a length of 0.500 mm.

The gel patch comprised a foam, double adhesive ring (diameter 3.8 cm, thickness 0.16 cm) having 0.35 mL of a hydrogel formulation and a skin contact area of 2 cm². The hydrogel formulation included tritiated PPS at 50 mg/mL in a 2% hydroxyethyl cellulose (HEC, NATROSOL® 250 HHX PHARM, HERCULES Int. Lim. Netherlands, determined molecular weight: Mw 1890000, Mn 1050000) hydrogel.

The gel patch was applied immediately following skin pretreatment with the pretreatment patch that had an area of 2 cm².

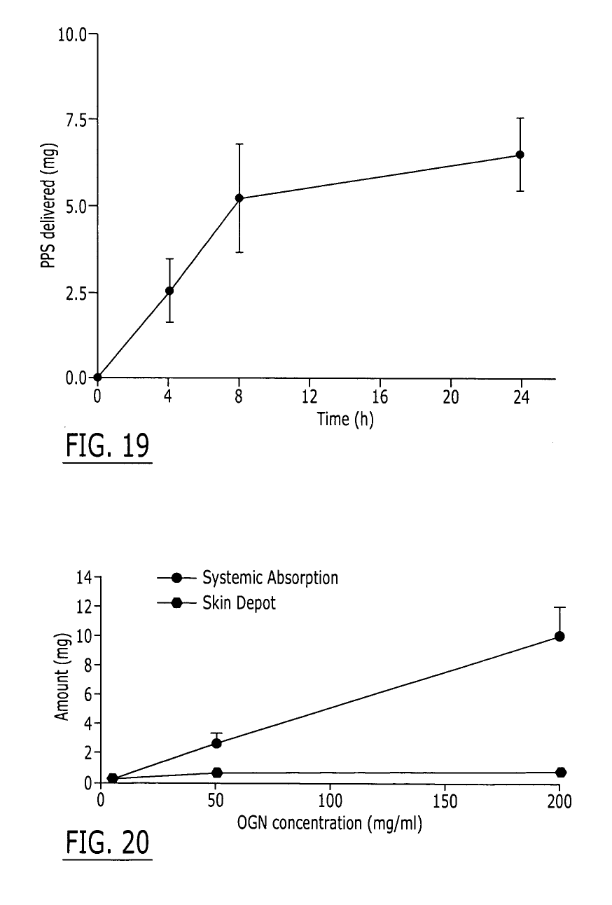

The amount of the biologically active agent (or drug) that penetrated the skin during selected time intervals was determined by measuring urinary excretion of tritium (previous studies had shown that in HGP's 32% of 3H-PPS injected intravenously is excreted in urine). The results indicated a time dependant flux of PPS through the skin (see FIG. 19). After 24 h delivery, more than 6 mg of PPS had been administered systemically.

### Example 2

As is also well known in the art, oligonucleotides are highly negatively charged compounds that do not penetrate the skin significantly without the use of penetration enhancers or physical disruption of the skin barrier. In this experiment, a 20-mer phosphorothioated oligonucleotide (OGN) was delivered by passive diffusion through pathways in the skin created by a pretreatment device of the invention.

The microprojection array comprised a stainless steel sheet having a thickness of 0.025 mm, an area of 2 cm², trapezoidally shaped microprojections bent at an angle of approximately 90° to the plane of the sheet and a microprojection density of 241 microprojections/cm². The microprojections had a length of 0.500 mm.

The gel patch comprised a foam, double adhesive ring (diameter 3.8 cm, thickness 0.16 cm) having 0.35 mL of a hydrogel formulation and a skin contact area of 2 cm². The hydrogel formulation included tritiated OGN at 5, 50, and 200 mg/mL in a 2% HEC (NATROSOL® 250 HHX) hydrogel.

The gel patch was applied immediately following skin pretreatment with the pretreatment patch.

At 24 hours after application, 3 systems from each group were removed and residual drug washed from the skin. The amount of the biologically active agent that penetrated the skin during prescribed time intervals was determined by measuring OGN liver content (previous studies had shown that following systemic administration in HGP's about 50% of the OGN accumulates in the liver). In addition, OGN skin content was also evaluated.

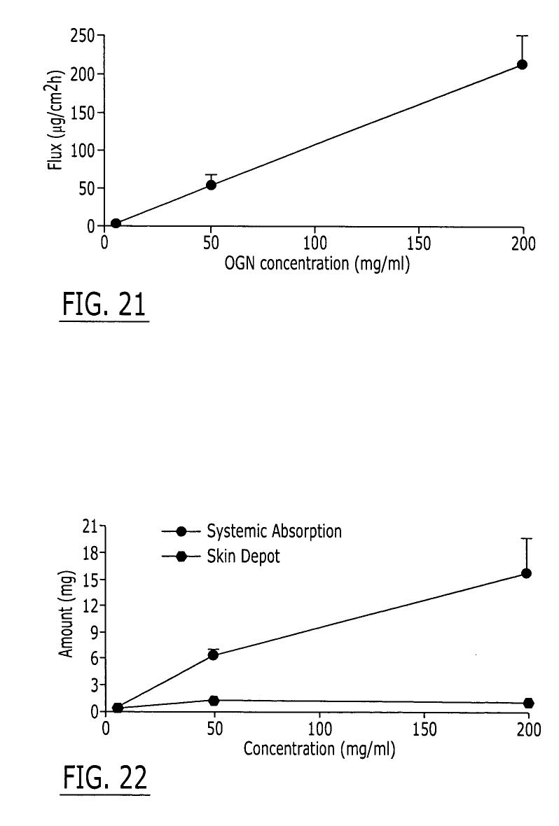

The results indicated a concentration dependant flux of the OGN through the skin (see FIGS. 20 and 21). At the highest concentration, a total of 10 mg had been absorbed systemically, corresponding to a drug utilization rate of 13.5%. At all concentrations, the skin depot was only a fraction of the systemic absorption.

### Example 3

An experiment similar to Example 2 above was performed using iontophoresis as the driving force in addition to passive diffusion. This was accomplished by inserting a silver chloride cathode between the backing membrane of the drug patch and the formulation containing the OGN. The system also comprised a silver foil anode, which was in contact with a saline reservoir gel. The electrodes were connected to a DC power source which supplied a constant level of electric current of 0.1 mA/cm².

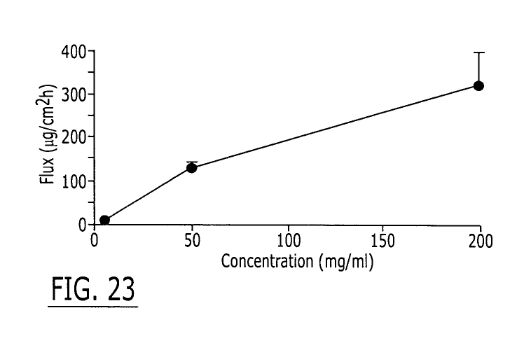

The results indicated a concentration dependant flux of the OGN through the skin (see FIGS. 22 and 23). At the highest concentration, a total of 15.6 mg had been absorbed systemically, corresponding to a drug utilization rate of 20.5%. At all concentrations, the skin depot was only a fraction of the systemic absorption.

From the foregoing description, one of ordinary skill in the art can easily ascertain that the present invention, among other things, provides an effective and efficient means for enhancing and extending the transdermal delivery of biologically active agents to a patient.

As will be appreciated by one having ordinary skill in the art, the present invention provides many advantages, such as:
Defined or regulated pretreatment area.
Defined or regulated pretreatment force and, hence, penetration into the stratum corneum.
Extended delivery profiles of biologically active agents.

Without departing from the spirit and scope of this invention, one of ordinary skill can make various changes and modifications to the invention to adapt it to various usages and conditions. As such, these changes and modifications are properly, equitably, and intended to be, within the full range of equivalence of the following claims.

1. A drug delivery system for delivering a biologically active agent through the skin of a patient, comprising:
a pretreatment patch adapted to be placed on the patient's skin, said pretreatment patch having a backing membrane ring and a microprojection array, said microprojection array being adhered to said backing membrane ring, said microprojection array including a plurality of microprojections adapted to pierce the stratum corneum of the patient, said pretreatment patch including a release liner ring that is removably secured to said backing membrane and a skin adhesive ring that is adhered to said release liner ring, said release liner ring and said skin adhesive ring being adapted to form a skin template on the patient's skin after said pretreatment patch is applied to and removed from the patient's skin; and
a gel patch having a top and bottom surface, said gel patch including a reservoir containing a hydrogel formulation, said gel patch having a skin contact area in the range of approximately 0.5-30 cm², said gel patch being adapted to be disposed on said skin template.

2. The delivery system of claim 1, wherein said gel patch includes a formulation membrane that is disposed proximate said gel patch reservoir, said formulation membrane being adapted to inhibit migration of enzymes into said hydrogel formulation.

3. The delivery system of claim 2, wherein said formulation membrane is adapted to inhibit migration of bacteria into said hydrogel formulation.

4. The delivery system of claim 1, wherein said pretreatment patch includes a polymeric membrane ring that is disposed between said release liner ring and said skin adhesive ring.

5. The delivery system of claim 4, wherein said skin template comprises said release liner ring, polymeric membrane ring and skin adhesive ring.

6. The delivery system of claim 1, wherein said pretreatment patch includes at least one release tag in communication with said release liner ring.

7. The delivery system of claim 1, wherein said pretreatment patch includes a polymeric membrane disposed between said backing membrane ring and said microprojection array.

8. The delivery system of claim 1, wherein said microprojection array has a microprojection density in the range of 10-2000 microprojections/cm².

9. The delivery system of claim 1, wherein said microprojection array provides a pretreated skin area in the range of approximately 0.5-30 cm² after said pretreatment patch is applied to the skin of the patient.

10. The delivery system of claim 1, wherein said pretreated skin area is substantially equal to said gel patch skin contact area.

11. The delivery system of claim 1, wherein said pretreated skin area is greater than said gel patch skin contact area.

12. The delivery system of claim 1, wherein said hydrogel formulation comprises a water-based hydrogel.

13. The delivery system of claim 12, wherein said hydrogel formulation comprises a polymeric material.

14. The delivery system of claim 13, wherein said polymeric material comprises a cellulose derivative.

15. The delivery system of claim 13, wherein said polymeric material is selected from the group consisting of EHEC, CMC, poly(vinyl alcohol), poly(ethylene oxide), poly(2-hydroxyethylmethacrylate), poly(n-vinyl pyrolidone) and mixtures thereof.

16. The delivery system of claim 1, wherein said hydrogel formulation includes at least one biologically active agent.

17. The delivery system of claim 16, wherein said biologically active agent is selected from the group consisting of polypeptides, proteins, oligonucleotides, nucleic acids and polysaccharides.

18. The delivery system of claim 16, wherein said biologically active agent is selected from the group consisting of a leutinizing hormone releasing hormone (LHRH), LHRH analogs, vasopressin, desmopressin, corticotropin (ACTH), ACTH analogs, including ACTH (1-24), calcitonin, parathyroid hormone (PTH), vasopressin, deamino [Val4, D-Arg8] arginine vasopressin, interferon alpha, interferon beta, interferon gamma, erythropoietin (EPO), granulocyte macrophage colony stimulating factor (GM-CSF), granulocyte colony stimulating factor (G-CSF), interleukin-10 (IL-10), glucagon, growth hormone releasing hormone (GHRH), growth hormone releasing factor (GHRF), insulin, insultropin, calcitonin, octreotide, endorphin, TRN, N-[[(s)-4-oxo-2-azetidinyl]carbonyl]-L-histidyl-L-prolinamide, liprecin, pituitary hormones, including HGH, HMG and desmopressin acetate, follicle luteoids, aANF, growth factors, including growth factor releasing factor (GFRF), bMSH, GH, somatostatin, bradykinin, somatotropin, platelet-derived growth factor releasing factor, asparaginase, bleomycin sulfate, chymopapain, cholecystokinin, chorionic gonadotropin, corticotropin (ACTH), erythropoietin, epoprostenol (platelet aggregation inhibitor), glucagon, HCG, hirulog, hyaluronidase, interferon, interleukins, menotropins (urofollitropin (FSH) and LH), oxytocin, streptokinase, tissue plasminogen activator, urokinase, vasopressin, desmopressin, ANP, ANP clearance inhibitors, BNP, VEGF, angiotensin II antagonists, antidiuretic hormone agonists, bradykinn antagonists, ceredase, CSI's, calcitonin gene related peptide (CGRP), enkephalins, FAB fragments, IgE peptide suppressors, IGF-1, neurotrophic factors, colony stimulating factors, parathyroid hormone and agonists, parathyroid hormone antagonists, prostaglandin antagonists, pentigetide, protein C, protein S, renin inhibitors, thymosin alpha-1, thrombolytics, TNF, vasopressin antagonists analogs, alpha-1 antitrypsin (recombinant), TGF-beta, and mixtures thereof.

19. The delivery system of claim 1, wherein said hydrogel formulation includes at least one pathway patency modulator.

20. The delivery system of claim 1, wherein said hydrogel formulation includes a vasoconstrictor.

21. The delivery system of claim 1, wherein said delivery system includes an applicator retainer that is adapted to cooperate with a pretreatment patch applicator.

22. The delivery system of claim 21, wherein said retainer includes a pretreatment patch seat adapted to receive said pretreatment patch.

23. The delivery system of claim 22, wherein said backing membrane ring includes adhesive tabs adapted to adhere to said pretreatment patch seat.

24. The delivery system of claim 23, wherein said pretreatment patch includes a supplemental adhesive ring that is adapted to cooperate with said skin adhesive ring, said supplemental adhesive ring being disposed between said release liner ring and said skin adhesive ring.

25. The delivery system of claim 23, wherein said retainer includes a pretreatment patch ring that is adapted to receive said pretreatment patch adhesive tabs during application of said pretreatment patch to the patient's skin, whereby said pretreatment patch is removable from the patient's skin by removing said retainer therefrom and whereby said skin template is disposed on the patient's skin.

26. The delivery system of claim 22, wherein said backing membrane ring includes a plurality of slots disposed proximate the periphery of said backing membrane ring and a plurality of break-away tabs adapted to cooperate with said pretreatment patch seat.

27. The delivery system of claim 26, wherein said retainer includes a pretreatment patch member having a plurality of posts that are adapted to engage said pretreatment patch slots during application of said pretreatment patch to the patient's skin, whereby said pretreatment patch is removable from the patient's skin by removing said retainer therefrom and whereby said skin template is disposed on the patient's skin.

28. A pretreatment device for pre-treating a patient's skin, comprising:
a backing membrane ring; and
a microprojection array, said microprojection array being adhered to said backing membrane ring, said microprojection array including a plurality of microprojections adapted to pierce the stratum corneum of the patient, said pretreatment patch including a release liner ring that is removably secured to said backing membrane and a skin adhesive ring that is adhered to said release liner ring, said release liner ring and said skin adhesive ring being adapted to form a skin template on the patient's skin after said pretreatment patch is applied to and removed from the patient's skin.

29. The pretreatment device of claim 28, wherein said pretreatment patch includes a polymeric membrane ring that is disposed between said release liner ring and said skin adhesive ring.

30. The pretreatment device of claim 29, wherein said skin template comprises said release liner ring, polymeric membrane ring and skin adhesive ring.

31. The pretreatment device of claim 28, wherein said pretreatment patch includes at least one release tag in communication with said release liner ring.

32. The pretreatment device of claim 28, wherein said pretreatment patch includes a polymeric membrane disposed between said backing membrane ring and said microprojection array.

33. The pretreatment device of claim 28, wherein said microprojection array has a microprojection density in the range of 10-2000 microprojections/cm².

34. The pretreatment device of claim 28, wherein said microprojection array provides a treated skin area in the range of approximately 0.5-30 cm² after said pretreatment patch is applied to the skin of the patient.

35. A method for delivering a biologically active agent through the skin of a patient, comprising the steps of:
providing a pretreatment patch adapted to be placed on the patient's skin, said pretreatment patch having a backing membrane ring and a microprojection array, said microprojection array being adhered to said backing membrane ring, said microprojection array including a plurality of microprojections adapted to pierce the stratum corneum of the patient, said pretreatment patch including a release liner ring that is removably secured to said backing membrane and a skin adhesive ring that is adhered to said release liner ring, said release liner ring and said skin adhesive ring being adapted to form a skin template on the patient's skin after said pretreatment patch is applied to and removed from the patient's skin;
providing a gel patch having a top and bottom surface, said gel patch including a reservoir containing a hydrogel formulation, said gel patch having a skin contact area in the range of approximately 0.5-30 cm²;
applying said pretreatment patch to the patient's skin, whereby said microprojections pierce said stratum corneum of the patient to provide a pretreated skin area having a plurality of microslits and whereby said skin template adheres to the patient's skin;
removing said pretreatment patch from the patient's skin; and
applying said gel patch to said pretreated skin area, said gel patch being positioned over said skin template, whereby said hydrogel formulation is released from said reservoir and migrates into and through said microslits formed in said stratum corneum by said pretreatment patch.

36. The method of claim 35, wherein said gel patch includes a formulation membrane that is disposed proximate said gel patch reservoir, said formulation membrane being adapted to inhibit migration of enzymes into said hydrogel formulation.

37. The method of claim 36, wherein said formulation membrane is adapted to inhibit migration of bacteria into said hydrogel formulation.

38. The method of claim 35, wherein said pretreatment patch includes a polymeric membrane ring that is disposed between said release liner ring and said skin adhesive ring.

39. The method of claim 38, wherein said skin template comprises said release liner ring, polymeric membrane ring and skin adhesive ring.

40. The method of claim 35, wherein said pretreatment patch includes at least one release tag in communication with said release liner ring.

41. The method of claim 35, wherein said pretreatment patch includes a polymeric support membrane disposed between said backing membrane ring and said microprojection array.

42. The method of claim 35, wherein said microprojection array has a microprojection density in the range of 10-2000 microprojections/cm².

43. The method of claim 35, wherein said a pretreated skin area is in the range of approximately 0.5-30 cm².

44. The method of claim 35, wherein said pretreated skin area is substantially equal to said gel patch skin contact area.

45. The method of claim 35, wherein said pretreated skin area is greater than said gel patch skin contact area.

46. The method of claim 35, wherein said hydrogel formulation comprises a water-based hydrogel.

47. The method of claim 46, wherein said hydrogel formulation comprises a polymeric material.

48. The method of claim 47, wherein said polymeric material comprises a cellulose derivative.

49. The method of claim 47, wherein said polymeric material is selected from the group consisting of EHEC, CMC, poly(vinyl alcohol), poly(ethylene oxide), poly(2-hydroxyethylmethacrylate), poly(n-vinyl pyrolidone) and mixtures thereof.

50. The method of claim 35, wherein said hydrogel formulation includes at least one biologically active agent.

51. The method of claim 50, wherein said biologically active agent is selected from the group consisting of polypeptides, proteins, oligonucleotides, nucleic acids and polysaccharides.

52. The method of claim 50, wherein said biologically active agent is selected from the group consisting of a leutinizing hormone releasing hormone (LHRH), LHRH analogs, vasopressin, desmopressin, corticotropin (ACTH), ACTH analogs, including ACTH (1-24), calcitonin, parathyroid hormone (PTH), vasopressin, deamino [Val4, D-Arg8] arginine vasopressin, interferon alpha, interferon beta, interferon gamma, erythropoietin (EPO), granulocyte macrophage colony stimulating factor (GM-CSF), granulocyte colony stimulating factor (G-CSF), interleukin-10 (IL-10), glucagon, growth hormone releasing hormone (GHRH), growth hormone releasing factor (GHRF), insulin, insultropin, calcitonin, octreotide, endorphin, TRN, N-[[(s)-4-oxo-2-azetidinyl]carbonyl]-L-histidyl-L-prolinamide, liprecin, pituitary hormones, including HGH, HMG and desmopressin acetate, follicle luteoids, aANF, growth factors, including growth factor releasing factor (GFRF), bMSH, GH, somatostatin, bradykinin, somatotropin, platelet-derived growth factor releasing factor, asparaginase, bleomycin sulfate, chymopapain, cholecystokinin, chorionic gonadotropin, corticotropin (ACTH), erythropoietin, epoprostenol (platelet aggregation inhibitor), glucagon, HCG, hirulog, hyaluronidase, interferon, interleukins, menotropins (urofollitropin (FSH) and LH), oxytocin, streptokinase, tissue plasminogen activator, urokinase, vasopressin, desmopressin, ANP, ANP clearance inhibitors, BNP, VEGF, angiotensin II antagonists, antidiuretic hormone agonists, bradykinn antagonists, ceredase, CSI's, calcitonin gene related peptide (CGRP), enkephalins, FAB fragments, IgE peptide suppressors, IGF-1, neurotrophic factors, colony stimulating factors, parathyroid hormone and agonists, parathyroid hormone antagonists, prostaglandin antagonists, pentigetide, protein C, protein S, renin inhibitors, thymosin alpha-1, thrombolytics, TNF, vasopressin antagonists analogs, alpha-1 antitrypsin (recombinant), TGF-beta, and mixtures thereof.

53. The method of claim 35, wherein said hydrogel formulation includes at least one pathway patency modulator.

54. The method of claim 35, wherein said hydrogel formulation includes a vasoconstrictor.

55. The method of claim 35, wherein said method includes providing an applicator retainer that is adapted to cooperate with a retainer patch applicator.

56. The method of claim 55, wherein said retainer includes a pretreatment patch seat adapted to receive said pretreatment patch.

57. The method of claim 56, wherein said backing membrane ring includes adhesive tabs adapted to adhere to said pretreatment patch seat.

58. The method of claim 57, wherein said pretreatment patch includes a supplemental adhesive ring that is adapted to cooperate with said skin adhesive ring, said supplemental adhesive ring being disposed between said release liner ring and said skin adhesive ring.

59. The method of claim 55, wherein said retainer includes a pretreatment patch ring that is adapted to receive said pretreatment patch adhesive tabs during said application of said pretreatment patch to the patient's skin, whereby said pretreatment patch is removable from the patient's skin by removing said retainer therefrom and whereby said skin template is adhered on the patient's skin.

60. The method of claim 55, wherein said backing membrane ring includes a plurality of slots disposed proximate the periphery of said backing membrane ring and a plurality of break-away tabs that are adapted to cooperate with said pretreatment patch seat.

61. The method of claim 60, wherein said retainer includes a pretreatment patch member having a plurality of posts that are adapted to engage said pretreatment patch slots during said application of said pretreatment patch to the patient's skin, whereby said pretreatment patch is removable from the patient's skin by removing said retainer therefrom and whereby said skin template is adhered to the patient's skin.

62. The method of claim 55, including the step of delivering up to 50 mg per day of said biologically active agent.

63. The method of claim 62, wherein said delivery step comprises zero-order delivery.)
